(19) United States
(12) Patent Application Publication
(10) Pub. No.: US 2015/0313921 A1
(43) Pub. Date: Nov. 5, 2015

(54) Cancer Treatment Methods

(71) Applicant: PLEDPHARMA AB, Stockholm (SE)

(72) Inventors: Jan Olof Karlsson, Trondheim (NO); Tino Kurz, Linköping (SE); Rolf Andersson, Vikingstad (SE)

(21) Appl. No.: 14/439,471

(22) PCT Filed: Oct. 31, 2013
(86) PCT No.: PCT/IB2013/059818

§ 371 Date: Apr. 29, 2015

Related U.S. Application Data
(60) Provisional application No. 61/721,570, filed on Nov. 2, 2012.

(51) Int. Cl. A61K 31/675; A61K 45/06

(52) U.S. Cl. CPC A61K 31/675; A61K 45/06

(57) ABSTRACT

Methods for treatment of cancer selected from lung cancer, ovarian cancer, squamous cell carcinoma, pancreas exocrine cancer, malignant melanoma, gastric cancer, esophageal cancer, a metastases thereof, and leukemia, in a human or non-human body, comprise administrating to the body a cancer-inhibiting amount of a first compound of Formula (I): or a physiologically acceptable salt thereof, wherein X, R¹, R², R³ and R⁴ are as defined herein.

### FIELD OF THE INVENTION

The present invention is directed to methods for treatment of cancer selected from lung cancer, ovarian cancer, squamous cell carcinoma, pancreas exocrine cancer, malignant melanoma, gastric cancer, esophageal cancer, a metastases thereof, and leukemia, in a human or non-human body. The methods comprise administrating to the body a cancer-inhibiting amount of a first compound of Formula I as defined herein.

### BACKGROUND OF THE INVENTION

EP 0910360, U.S. Pat. No. 6,147,094, EP 0936915, U.S. Pat. No. 6,258,828, EP 1054670, U.S. Pat. No. 6,310,051, EP 1060174, and U.S. Pat. No. 6,391,895 disclose the use of dipyridoxyl based chelating agents and their metal chelates and the use of certain manganese containing compounds, in particular manganese chelates, in medicine. The use of such compounds as cell protective agents in cancer therapy is also disclosed. The above cited documents disclose that certain chelating agents, in particular dipyridoxyl and aminopolycarboxylic acid-based chelating agents and their metal chelates are effective in treating or preventing anthracycline-induced cardiotoxicity, ischemia-reperfusion-induced injuries and atherosclerosis. Dipyridoxyl based chelating agents and their chelates with trivalent metals have previously been described by Taliaferro (Inorg. Chem. 1984; 23:1183-1192).

DPDP (N,N′-bis-(pyridoxal-5-phosphate)-ethylenediamine-N,N′-diacetic acid), and the dephosphorylated counterpart PLED (N,N′-dipyridoxyl ethylenediamine-N,N′-diacetic acid) are dipyridoxyl compounds capable of chelating metals. It has previously been described that the manganese chelates of these compounds, MnDPDP and its dephosphorylated counterpart MnPLED, possess catalytic antioxidant activity, i.e., a superoxide dismutase (SOD) mimetic activity. These compounds have been shown to have a protective effect in normal cells, e.g., against the cytostatic drug doxorubicin and ischemia-reperfusion. It is the SOD mimetic activity, which is an inherent property of redox-active manganese (Mn²⁺/Mn³⁺) bound to DPDP/PLED (Brurok et al., Biochem Biophys Res Commun. 1999; 254:768-721), that explains the protective effects. Consequently, Brurok and co-workers (1999) have shown that the PLED metal complex loses its catalytic activity after replacing redox-active manganese with redox-inactive zinc (Zn²⁺).

Laurent et al. (Cancer Res. 2005; 6:948-56) and Alexandre et al., (J Natl Cancer Inst. 2006; 98:236-44) have recently described that MnDPDP (equivalent to the ready-to-use MRI contrast agent Teslascan) not only increased survival of normal cells but also increased cancer cell death during cytostatic treatment, e.g., with oxaliplatin. Cytostatic drugs may cause cancer cell death by elevating intracellular H₂O₂ and inducing apoptosis. The Laurent et al. hypothesis was that MnDPDP, due to its SOD mimetic activity, elevated intracellular H₂O₂ and hence acted in synergy with cytostatic drugs. Since the basal level of H₂O₂ is much lower in normal cells compared to cancer cells, the authors suggested that elevation from a low H₂O₂ level induced cell survival in normal cells. They furthermore suggested that elevation from a much higher basal level of H₂O₂ in cancer cells at the same time resulted in apoptotic signaling and hence cell death. Consequently, these authors suggested that both these effects, i.e., the increase in cancer cell death and survival of normal cells, were caused by the SOD mimetic activity of MnDPDP, an activity which is absolutely dependent on redox-active manganese. It has also been shown that intravenous injection of both the mother compound MnDPDP and its metabolite MnPLED into mice gave rise to protection against certain cytostatic drugs (EP 0910360 and U.S. Pat. No. 6,147,094). When MnDPDP is intravenously injected into humans, about 80% of the administered manganese is released. For diagnostic imaging use and for occasional therapeutic use, dissociation of manganese from MnDPDP represents no major problem. However, for more frequent use, accumulated manganese toxicity may represent a serious toxicological problem, particularly when it comes to neurotoxicity (Crossgrove & Zheng; NMR Biomed. 2004; 17:544-53). Thus, for frequent therapeutic use, as in cancer treatment, compounds that dissociate manganese should be avoided.

A number of anti-tumour agents are associated with adverse side effects. Paclitaxel, for example, is one such cytostatic drug which has shown anti-neoplastic activity against a variety of malignant tissues, including those of the breast. However, at the dosages required to have an anti-neoplastic effect, paclitaxel has a number of adverse side-effects which include cardiovascular irregularities as well as hematological and gastrointestinal toxicity. Oxaliplatin, in particular in combination with 5-fluorouracil (5-FU), is another example of a cytostatic drug that is effective in the treatment of colorectal cancer but its use is restricted by severe adverse side-effects, in particular, hematological toxicity and neurotoxicity. Severe side-effects also restrict the use of radiation therapy in cancer.

There is hence an unmet medical need to find new chemotherapeutic drugs with fewer side-effects, in addition to finding methods to protect normal cells against injuries caused by cancer treatment.

### SUMMARY OF THE INVENTION

The present invention overcomes various deficiencies of the prior art. In one embodiment, the invention is directed to a method of treatment of cancer selected from lung cancer, ovarian cancer, squamous cell carcinoma, pancreas exocrine cancer, malignant melanoma, gastric cancer, esophageal cancer, a metastases thereof, and leukemia, in a human or non-human body. The method comprises administrating to the body a cancer-inhibiting amount of a first compound of Formula I:

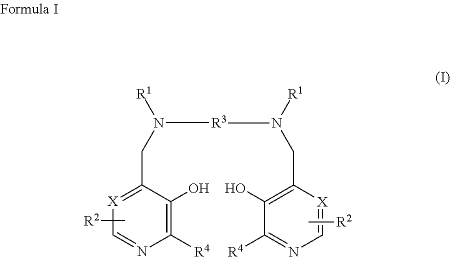

or a physiologically acceptable salt thereof, wherein

### X is CH or N,

each R¹ independently is hydrogen or —CH₂COR⁵;
R⁵ is hydroxy, ethylene glycol, glycerol, optionally hydroxylated alkoxy, amino or alkylamido;
each R² independently is a group ZYR⁶;
Z is a bond, CO, or a C₁₋₃ alkylene or oxoalkylene group optionally substituted by a group R⁷;
Y is a bond, an oxygen atom or a group NR⁶;
R⁶ is a hydrogen atom, COOR⁸, an alkyl, alkenyl, cycloalkyl, aryl or aralkyl group optionally substituted by one or more groups selected from COOR⁸, CONR⁸₂, NR⁸₂, OR⁸, ═NR⁸, ═O, OP(O)(OR⁸)R⁷ and OSO₃M;
R⁷ is hydroxy, an optionally hydroxylated, optionally alkoxylated alkyl or aminoalkyl group;
R⁸ is a hydrogen atom or an optionally hydroxylated, optionally alkoxylated alkyl group;
M is a hydrogen atom or one equivalent of a physiologically tolerable cation;
R³ is a C₁₋₈ alkylene group, a 1,2-cykloalkylene group, or a 1,2-arylene group, optionally substituted with R⁷; and
each R⁴ independently is hydrogen or C₁₋₃ alkyl.

The methods of the invention provide advantageous therapeutic treatment. These and additional advantages and embodiments of the invention will be more fully apparent in view of the detailed description.

### BRIEF DESCRIPTION OF THE DRAWINGS

The Detailed Description will be more fully understood in view of the Drawings, in which

FIG. 1 shows the cytotoxic activity of DPDP and MnDPDP toward non-small cell lung cancer (NSCLC) U1810 cells in the method described in Example 1 (mean±SD; n=3);

FIG. 2 shows the cytotoxic activity of Doxorubicin (Dx) alone on A2780 cancer cells (mean±SD; n=3), described in Example 2;

FIG. 3 shows the cytotoxic activity of a wider range of concentrations of Doxorubicin (Dx) alone on A2780 cancer cells (mean±SD; n=3), described in Example 2;

FIG. 4 shows the cytotoxic activity of MnDPDP in A2780 cancer cells in the presence and absence of varying concentrations of Doxorubicin (Dx) (mean±SD; n=3), described in Example 2;

FIG. 5 shows the cytotoxic activity of DPDP in A2780 cancer cells in the presence and absence of varying concentrations of Doxorubicin (Dx) (mean±SD; n=3)), described in Example 2; and

FIG. 6 shows the cytotoxic activity of Dx in A2780 cancer cells at varying concentrations of DPDP (mean±SD; n=3), described in Example 2.

### DETAILED DESCRIPTION

In one embodiment, the invention is directed to a method of treatment of cancer selected from lung cancer, ovarian cancer, squamous cell carcinoma, pancreas exocrine cancer, malignant melanoma, gastric cancer, esophageal cancer, a metastases thereof, and leukemia in a human or non-human body. The method comprises administrating to the body a cancer-inhibiting amount of a first compound of Formula I:

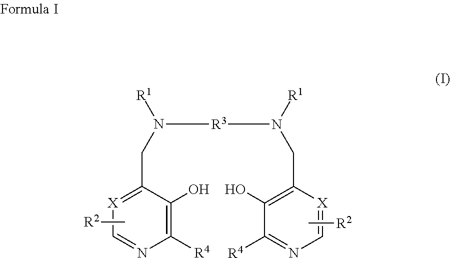

or a physiologically acceptable salt thereof, wherein

### X is CH or N,

each R¹ independently is hydrogen or —CH₂COR⁵;
R⁵ is hydroxy, ethylene glycol, glycerol, optionally hydroxylated alkoxy, amino or alkylamido;
each R² independently is a group ZYR⁶;
Z is a bond, CO, or a C₁₋₃ alkylene or oxoalkylene group optionally substituted by a group R⁷;
Y is a bond, an oxygen atom or a group NR⁶;
R⁶ is a hydrogen atom, COOR⁸, an alkyl, alkenyl, cycloalkyl, aryl or aralkyl group optionally substituted by one or more groups selected from COOR⁸, CONR⁸₂, NR⁸₂, OR⁸, ═NR⁸, ═O, OP(O)(OR⁸)R⁷ and OSO₃M;
R⁷ is hydroxy, an optionally hydroxylated, optionally alkoxylated alkyl or aminoalkyl group;
R⁸ is a hydrogen atom or an optionally hydroxylated, optionally alkoxylated alkyl group;
M is a hydrogen atom or one equivalent of a physiologically tolerable cation;
R³ is a C₁₋₈ alkylene group, a 1,2-cykloalkylene group, or a 1,2-arylene group, optionally substituted with R⁷; and
each R⁴ independently is hydrogen or C₁₋₃ alkyl.

The compounds of Formula I as defined above for use in the invention should be understood to be therapeutically active and physiologically acceptable compounds.

As used herein the terms “alkyl” and “alkylene” include straight-chained and branched, saturated and unsaturated hydrocarbons. The term “1,2-cykloalkylene” includes both cis and trans cycloalkylene groups and alkyl substituted cycloalkylene groups having from 5-8 carbon atoms. The term “1,2-arylene” includes phenyl and naphthyl groups and alkyl substituted derivatives thereof having from 6 to 10 carbon atoms.

Unless otherwise specified, any alkyl, alkylene or alkenyl moiety may conveniently contain from 1 to 20, specifically 1-8, more specifically 1-6, and, even more specifically, 1-4 carbon atoms.

Cycloalkyl, aryl and aralkyl moieties may conveniently contain 3-18, specifically 5-12, and more specifically 5-8 ring atoms. Aryl moieties comprising phenyl or naphthyl groups are preferred. Specific aralkyl groups include, but are not limited to, phenyl C₁₋₈ alkyl, and, more specifically, benzyl.

Where groups may optionally be substituted by hydroxyl groups, this may be monosubstitution or polysubstitution and, in the case of polysubstitution, alkoxy and/or hydroxyl substituents may be carried by alkoxy substituents.

In specific embodiments of the compound of Formula I, each group R¹ represents —CH₂COR⁵, R³ is a C₁₋₆ alkylene group or, more specifically, a C₂₋₄ alkylene group, and/or R⁵ is hydroxyl, C₁₋₈ alkoxy, ethylene glycol, glycerol, amino or C₁₋₈ alkylamido. In a more specific embodiment, each group R¹ represents —CH₂COR⁵ in which R⁵ is hydroxy.

In further specific embodiments of the compound of Formula I, each R² is CHR⁷OCO(CH₂)ₓPh or CHR⁷OCO(CH₂CO)ₓ(Ph, in which x is 1 to 3, CHR⁷OCOBu, CH₂N(H)R^{6′}, CH₂N(H)R^{6′}, N(H)R^{6′}, N(R^{6′})₂, CH₂OH, CH₂OR^{6′}, COOR^{6′}, CON(H)R^{6′}, CON(R^{6′})₂ or OR^{6′}, in which R^{6′} is a mono- or polyhydroxylated alkyl group, specifically a C₁₋₄, or, more specifically, a C₁₋₃ alkyl group, (CH₂)ₙCOOR^{7′} in which n is 1 to 6 or COOR^{7′}, in which R^{7′} is a C₁₋₄ alkyl, specifically C₁₋₃ alkyl, or, more specifically, a methyl group, CH₂OSO₃⁻M, CH₂CH₂COOH, CH₂OP(O)(OH)(CH₂)₃NH₂, CH₂OP(O)(OH)CH₃ or CH₂OP(O)(OH)₂ group. In a more specific embodiment, each R² represents a group of the formula CH₂OP(O)(OH)₂.

The compound of Formula I may have the same or different R² groups on the two pyridyl rings and these may be attached at the same or different ring positions. In specific embodiments of the compound of Formula I, the R² group substitution is at the 5- and 6-positions, or, more specifically, at the 6-position, i.e. para to the hydroxyl group. In specific embodiments of the compound of Formula I, the R² groups are identical and identically located, e.g. 6,6′.

In further specific embodiments of the compound of Formula I, Z is a bond or a group selected from CH₂, (CH₂)₂, CO, CH₂CO, CH₂CH₂CO or CH₂COCH₂, and/or Y represents a bond.

In further specific embodiments of the compound of Formula I, each R⁶ is mono- or poly(hydroxy or alkoxylated) alkyl groups or a group of the formula OP(O)(OR⁸)R⁷ and/or R⁷ is hydroxyl or an unsubstituted alkyl or aminoalkyl group.

In further specific embodiments of the compound of Formula I, R³ is ethylene and R² has any of the R² identities listed above.

The compound is optionally a chelate with one or two Na⁺ or K⁺, but a combination of one Na⁺ and one K⁺ is also possible.

In one embodiment, R⁵ is hydroxy, C₁₋₈ alkoxy, ethylene glycol, glycerol, amino or C₁₋₈ alkylamido; Z is a bond or a group selected from CH₂, (CH₂)₂, CO, CH₂CO, CH₂CH₂CO or CH₂COCH₂; Y is a bond; R⁶ is a mono- or poly(hydroxy or alkoxylated) alkyl group or a group of the formula OP(O)(OR⁸)R⁷; and R⁷ is hydroxy, or an unsubstituted alkyl or aminoalkyl group.

In another embodiment of the invention, R³ is ethylene and each group R¹ represents —CH₂COR⁵ in which R⁵ is hydroxy.

In yet another embodiment of the invention, the compound of Formula I is N,N′-dipyridoxyl ethylenediamine-N,N′-diacetic acid (PLED).

In a further embodiment of the invention, the compound of Formula I is N,N′-bis-(pyridoxal-5-phosphate)-ethylenediamine-N,N′-diacetic acid (DPDP).

The invention thus comprises a method employing a compound of Formula I, and in a specific embodiment, the compound DPDP, or one of its dephosphorylated counterparts DPMP and PLED, representing a method for treating various cancer diseases, alone or in combination with a cyto-protective compound and/or other cytostatic drugs and/or radiotherapy, as described below. Specific embodiments are directed to the treatment of lung cancer, ovarian cancer, squamous cell carcinoma, pancreas exocrine cancer, malignant melanoma, gastric cancer, esophageal cancer, a metastases thereof (i.e., metastases of lung cancer, ovarian cancer, squamous cell carcinoma, pancreas exocrine cancer, malignant melanoma, gastric cancer, and/or esophageal cancer), and/or leukemia. Thus, a specific method is directed to treatment of lung cancer, or, more specifically, non-small cell lung cancer, and/or metastases thereof. Another specific method is directed to treatment of ovarian cancer and/or metastases thereof. Another specific method is directed to treatment of squamous cell carcinoma and/or metastases thereof. Another specific method is directed to treatment of pancreas exocrine cancer and/or metastases thereof. Another specific method is directed to treatment of malignant melanoma and/or metastases thereof. Another specific method is directed to treatment of gastric cancer and/or metastases thereof. Another specific method is directed to treatment of esophageal cancer and/or metastases thereof. Another specific method is directed to treatment of leukemia.

In another embodiment of the invention, a compound of Formula I, as defined above, is used in the manufacture of a medicament for treatment of cancer selected from lung cancer, ovarian cancer, squamous cell carcinoma, pancreas exocrine cancer, malignant melanoma, gastric cancer, esophageal cancer, a metastases thereof, and leukemia. Thus, a specific medicament is for treatment of lung cancer, or, more specifically, non-small cell lung cancer, and/or metastases thereof. Another specific medicament is for treatment of ovarian cancer and/or metastases thereof. Another specific medicament is for treatment of squamous cell carcinoma and/or metastases thereof. Another specific medicament is for treatment of pancreas exocrine cancer and/or metastases thereof. Another specific medicament is for treatment of malignant melanoma and/or metastases thereof. Another specific medicament is for treatment of gastric cancer and/or metastases thereof. Another specific medicament is for treatment of esophageal cancer and/or metastases thereof. Another specific medicament is for treatment of leukemia. The medicament may be in the form of a pharmaceutical composition, comprising one or more pharmaceutically acceptable carriers or excipients.

In the methods of treatment of the invention, a patient in need of such treatment is administered a cancer inhibiting amount of the compound of Formula (I), for example, in a pharmaceutical composition comprising one or more pharmaceutically acceptable carriers and/or excipients. The pharmaceutical compositions for use in the methods of the present invention may be formulated with conventional pharmaceutical or veterinary formulation aids, for example stabilizers, antioxidants, osmolality adjusting agents, buffers, pH adjusting agents, sweetening agents, etc.

The pharmaceutical compositions for use in the methods of the present invention may be in a conventional pharmaceutical administration form such as a tablet, capsule, powder, solution, suspension, dispersion, syrup, suppository, etc. In one specific embodiment, the pharmaceutical compositions for use in the methods of the present invention may be in a form suitable for parenteral or enteral administration, for example injection or infusion. The compounds of Formula I may, for example, be suspended or dissolved in an aqueous medium, optionally with the addition of pharmaceutically acceptable excipients. The compounds and the pharmaceutical compositions for use in the methods according to the present invention may be administered by various routes, for example orally, transdermally, rectally, intrathecally, topically, or by means of inhalation or injection, in particular subcutaneous, intramuscular, intraperitoneal or intravascular injection. Other routes of administration may be envisioned, for example, to increase the effectiveness, the bioavailability, and/or the tolerance of the compositions. The most appropriate route can be chosen by those skilled in the art according to the formulation used.

In an additional aspect of the invention, the methods comprise administering both a first compound of Formula I, as defined hereinabove, and a second compound having a cyto-protective ability. In a specific embodiment, the second compound is a metal chelate of a compound of Formula I as defined above. The first compound and the second compound may be administered in a single pharmaceutical composition or in separate compositions, such compositions optionally including one or more pharmaceutically acceptable carriers and/or excipients as discussed above, for administration by any of the various routes discussed above. When administered as separate compositions, the first compound and the second compound may be administered simultaneously, sequentially, or at separate times, to a patient in need thereof.

In yet another embodiment of the invention, the second compound comprises a metal chelate having a Kₐ value preferably in the range of from 10⁸ to 10²⁴, more specifically in a range of from 10¹⁰ to 10²² and, even more specifically, in the range of from 10¹² to 10²⁰. In a further embodiment of the invention, the metal chelate has a lower Kₐ value than the Kₐ value of an iron (Fe³⁺) chelate of a compound of Formula I as defined above, by a factor of at least 10³. In still another embodiment of the invention, the metal in the metal chelate is manganese (Mn²⁺ or Mn³⁺) or copper (Cu⁺ or Cu²⁺).

In another embodiment of the invention, the first compound is N,N′-dipyridoxyl ethylenediamine-N,N′-diacetic acid and the second compound is a metal chelate of N,N′-dipyridoxyl ethylenediamine-N,N′-diacetic acid. The metal in the metal chelate is preferably manganese or copper. In another embodiment of the invention the first compound is N,N′-bis-(pyridoxal-5-phosphate)-ethylenediamine-N,N′-diacetic acid and the second compound is a metal chelate of N,N′-dipyridoxyl ethylenediamine-N,N′-diacetic acid. The metal in the metal chelate is preferably manganese or copper.

If not all of the labile hydrogens of the chelates according to the invention are substituted by the complexed metal ion, biotolerability and/or solubility of the chelates may be increased by substituting the remaining labile hydrogen atoms with physiologically biocompatible cations of inorganic and/or organic bases or amino acids. Examples of suitable inorganic cations include Li⁺, K⁺, Na⁺ and Ca²⁺. Suitable organic cations include ammonium, substituted ammonium, ethanolamine, diethanolamine, morpholine, glucamine, N,N,-dimethyl glucamine, lysine, arginine or ornithine.

Additionally, where the first or the second compound according to the invention carries an overall charge, it may conveniently be used in the form of a salt with a physiologically acceptable counterion, for example an ammonium, substituted ammonium, alkali metal or alkaline earth metal (e.g. calcium) cation or an anion deriving from an inorganic or organic acid. In a specific embodiment, meglumine salts are employed.

In a further embodiment of the invention, the second compound is employed in an amount of 1/100 to 99/100 of the first compound, on a molar basis.

In a further embodiment of the invention, the compound of Formula (I) is administered together with one or more other anti-cancer drug(s). The anti-cancer drug may be any anticancer drug, examples of which include, but are not limited to, doxorubicin, epirubicin, oxaliplatin, cisplatin, carboplatin, paclitaxel, docetaxel, 5-fluorouracil, cyclophosphamide, gemcitabine, irinotecan, and methotrexate. The compound of Formula (I) and the one or more other anti-cancer drug(s) may be administered in a single pharmaceutical composition or in separate compositions, such compositions optionally including one or more pharmaceutically acceptable carriers and/or excipients as discussed above, for administration by any of the various routes discussed above. When administered as separate compositions, the first compound and the one or more other anti-cancer drug(s) may be administered simultaneously, sequentially, or at separate times, to a patient in need thereof.

Additionally, in a further embodiment, the first compound of Formula (I), the second compound, for example, the metal chelate of a compound of Formula (I), and one or more anticancer drugs, for example, as described above, are administered to a patient in need of treatment of cancer selected from lung cancer, ovarian cancer, squamous cell carcinoma, pancreas exocrine cancer, malignant melanoma, gastric cancer, esophageal cancer, a metastases thereof, and leukemia. The first compound of Formula (I), the second compound, for example, the metal chelate of a compound of Formula (I), and the one or more anticancer drugs may be administered in a single pharmaceutical composition or in separate compositions, such compositions optionally including one or more pharmaceutically acceptable carriers and/or excipients as discussed above, for administration by any of the various routes discussed above. When administered as separate compositions, the first compound, the second compound, and the one or more other anti-cancer drug(s) may be administered simultaneously, sequentially, or at separate times, to a patient in need thereof.

In a further embodiment of the invention, in a method of treatment as described in any of the aforementioned embodiments, the treatment is combined with radiation therapy.

The cancer inhibiting amount of a medicament administered to a patient is dependent on several different factors such as the type of cancer, the age and weight of the patient, etc., and the attending physician will follow the treatment to adjust the doses if necessary based on laboratory tests.

Generally doses of the active compounds, i.e., the first compound of Formula (I) and, optionally, the second compound, for example, a metal chelate of a compound of Formula (I), are in a range of 0.01 μmol of the compound per kilogram of the patient's body weight to 100 μmol of the compound per kilogram of the patient's body weight.

As previously described, the invention provides a compound of Formula I as defined above for use in the treatment of cancer. When the present inventors compared MnDPDP and DPDP they surprisingly found that DPDP was more efficacious than MnDPDP in its ability to kill cancer cells and they concluded that the previously described cancer cell killing ability of MnDPDP is an inherent property of DPDP. The invention thus provides a new method for treatment of cancer while avoiding the problem of toxicity related to manganese release.

The compound may, as previously mentioned, also be used in combination with a second compound having cyto-protective ability. In one embodiment of the invention, a metal chelate of a compound of Formula I is used as the compound having the cyto-protective ability. In the present methods, employing the metal chelate such as MnPLED is surprisingly found to be much more stable than MnDPDP alone and the problem of metal release is thereby avoided. A suitable drug combination for cancer-treatment is thus presented. The stability of MnDPDP after administration into man is according to prior art mainly governed by the stability constants between DPDP and Mn²⁺ and other competing metals, mainly non-redox active Zn²⁺ which has higher affinity for DPDP than Mn²⁺ (Rocklage et al., Inorg Chem 1989; 28:477-485 and Toft et al., Acta Radiol 1997; 38:677-689). After intravenous injection in man, in addition to dissociation of Mn²⁺, the two phosphates are hydrolyzed from DPDP, giving rise to PLED. Shortly after intravenous injection about 30% of the injected MnDPDP is transformed into MnPLED, and according to prior art (Toft et al., 1997), Mn²⁺ will also dissociate from PLED, actually more readily than from DPDP. Such behaviour of MnPLED is highly supported by the reported stability constants in the literature (Rocklage et al., 1989).

However, reinterpretation of previously published results may in fact suggest that MnPLED is much more stable than MnDPDP (regarding metal stability) during in vivo conditions. If human plasma concentration data taken from the study by Toft et al. 1997 is recalculated it is seen that disappearance of MnDPDP and its 5 metabolites from the plasma roughly parallels that of MnPLED between 30 and 60 minutes (after the initial distribution phase). All these compounds are eliminated from the body through renal excretion, and if manganese dissociated from MnPLED, one would expect that these two processes diverged during that period of time. This finding may suggest that MnPLED is stable during in vivo conditions. It may furthermore be anticipated that target cells and tissues will not be exposed for concentrations higher than 5 μM of MnPLED, i.e., concentrations where MnPLED are expected to be stable, and that MnPLED is much more stable than MnDPDP, and most importantly, by using MnPLED instead of its mother substance MnDPDP, it may be possible to circumvent the serious toxicological manganese problem evident at frequent therapeutic use in man.

It should furthermore be stressed that pretreatment with MnPLED in mice has shown to be approximately 100 times more efficacious than MnDPDP (EP 0910360 and U.S. Pat. No. 6,147,094). This suggests that the MnPLED dose could be considerable lowered in comparison to MnDPDP, which would further reduce the toxicological potential of the pharmaceutical composition, and hence increase the therapeutic index further. Moreover, a lower dose of MnPLED (3 μmol/kg) than that employed in MnDPDP-enhanced diagnostic imaging (5-10 μmol/kg) has been shown to reduce infarct size in pigs (Karlsson et al., Acta Radiol 2001; 42:540-547), and even much lower doses have been demonstrated to be effective in the same animal model (unpublished data). Interestingly, MnDPDP did not reduce the infarct size in pigs. This is presumably due to a much faster replacement of manganese for zinc in pigs compared to man. Ten minutes after injection of MnDPDP all manganese has been replaced with zinc (Karlsson et al., 2001), which differs from man (and some other investigated species) where about 30% of the injected manganese stays bound to the chelator for a considerable amount of time. As mentioned previously, the protection of normal cells, in this case myocardial cells, is dependent on redox-active manganese. According to prior art (Rocklage et al., 1989), the stability constant between Mn²⁺ and DPDP is 15.10 (logK), whereas the stability constant between Zn²⁺ and DPDP is 18.95, i.e., Mn²⁺ dissociates about 1000 times more readily than Zn²⁺ from DPDP. The corresponding stability constants between Mn²⁺ and PLED and Zn²⁺ and PLED are 12.56 and 16.68, respectively, i.e., Mn²⁺ once again dissociates about 1000 times more readily than Zn²⁺. From this and the published metabolic scheme (Toft et al., 1997) one would not expect any major difference in stability between MnDPDP and MnPLED, in respect to exchange of manganese for zinc, after administration into pigs. The above mentioned infarct reduction seen after administration of MnPLED, but not after MnDPDP, is hence a paradoxical finding. However, the present inventors explain that MnPLED is a more stable complex than MnDPDP, and, most importantly, it solves the toxicological problems of manganese instability.

An advantage of combining the DPDP's anticancer activity with MnPLED's cyto-protective activity with regard to normal cells and tissue may be exemplified by the problem of using dexrazoxane as a cardioprotective agent against anthracycline-induced cardiotoxicity. Although far from evident, dexrazoxane is not recommended at the beginning of the anthracycline therapy in patients with metastatic breast cancer because of the possibility of reducing the anticancer effect of the anthracyclines (Yeh et al., Circulation 2004; 109:3122-3132). However, as has been demonstrated for MnDPDP by the present inventors and others, preclinical data quite clearly shows that this is not a problem when it comes to our approach. One conceivable explanation to this is the two distinct and inherent activities of MnDPDP, namely its anticancer activity and its cytoprotective activity, and which in our invention has been further separated into two distinct chemical entities, namely DPDP, possessing the anticancer activity, and MnPLED, possessing the cytoprotective activity in normal cells and tissues.

### EXAMPLES

The invention will now be further demonstrated and described by the following non-limiting examples. The examples should be understood to only exemplify the invention and the invention should not be limited thereto.

### Example 1

The cytotoxic activity of DPDP toward human non-small cell lung cancer (NSCLC) U1810 cells was compared with that of MnDPDP.

### Methods

The viability of cells was measured using the MTT assay. Briefly, 8,000 human U1810 NSCLC cells were seeded per well on a 96-well plate and grown over night in RPMI (Roswell Park Memorial Institute) 1640 medium containing 10% fetal bovine serum, 2 mM L-glutamine, 100 UI/ml penicillin and 100 μg/ml streptomycin at 37° C. in humidified air with 5% CO₂. Cells were then exposed for 48 h to 1-1,000 μM DPDP (lot #RDL02090206) or MnDPDP (lot #02090106). The viability of the cells was then assessed by adding 5 mg/ml methylthiazoletetrazolium (MTT) to a final concentration of 0.5 mg/ml and incubating cells for a further 4 h at 37° C. The blue formazan that is formed by mitochondrial dehydrogenases of viable cells was then dissolved over night at 37° C. by adding 10% SDS and 10 mM HCl to a final concentration of 5% SDS and 5 mM HCl. Finally, the absorbance of the solution was read at 570 nm with a reference at 670 nm in a microplate reader Spectramax 340 (Molecular Devices, Sunnyvale, Calif., USA) connected to an Apple Macintosh computer running the program Softmax Pro V1.2.0 (Molecular Devices, Sunnyvale, Calif., USA). The viability of U1810 cells in the presence of increasing concentrations of DPDP or MnDPDP is presented as concentration-response curves (mean±S.D.). The individual curves were fitted to the sigmoidal normalized response logistic equation (Graphpad Prism, version 5.02). From this analysis the concentrations causing 50% inhibition (IC₅₀) of the test substances were calculated.

### Results

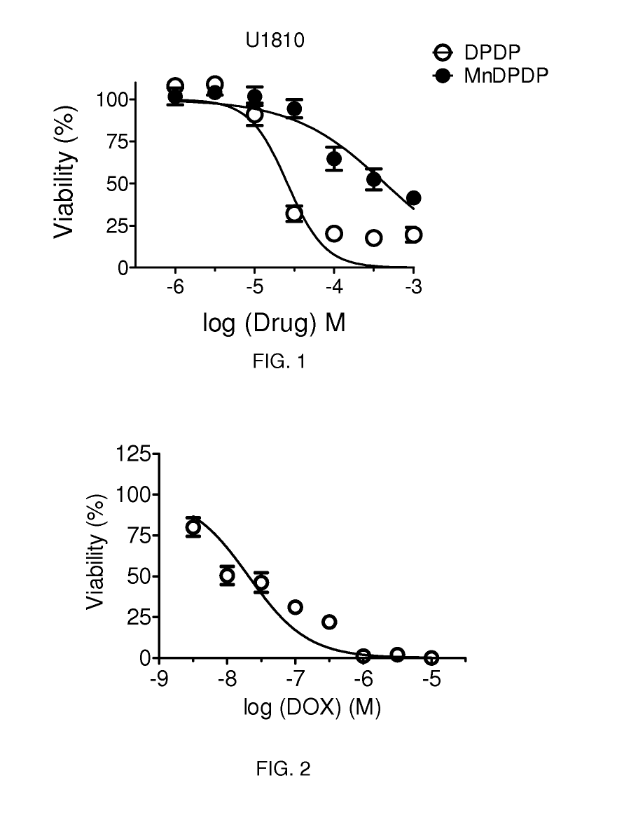

The cytotoxic activity of DPDP and MnDPDP toward NSCLC U1810 cells is shown in FIG. 1. The calculated IC₅₀ ratio (0.0004368/0.00005282) between MnDPDP and DPDP showed that DPDP was 17 times more potent than MnDPDP to kill U1810 cells.

### Conclusions

The present results show efficacy of the presently claimed treatment methods. These results also demonstrate that the previously described cytotoxic activity MnDPDP is an inherent property of the DPDP or its dephosphorylated counterparts and not of the intact metal complex MnDPDP. Dissociation of manganese to some extent from DPDP probably explains the cancer killing efficacy of MnDPDP.

### Example 2

The cytotoxic activity of DPDP and MnDPDP against human tumor line A2780 was compared.

### Methods

Specifically, the ovarian carcinoma A2780 has a tumor doubling time of approximately 2 days and is sensitive to doxorubicin (Dx). A2780 cells were co-incubated with MnDPDP, DPDP, and/or Dx. The viability of cells was measured using the methylthiazoletetrazolium (MTT) assay. Briefly, 8000 cells were seeded per well on a 96-well plate and grown overnight under standards. Cells were then exposed for 48 hours to various concentrations of MnDPDP, DPDP, and/or Dx at 37° C. The viability of the cells was then assessed by adding 5 mg/ml MTT to a final concentration of 0.5 mg/ml and incubating cells for a further 4 hours at 37° C. The blue formazan that is formed by mitochondrial dehydrogenases of viable cells was then dissolved overnight at 37° C. by adding 10% SDS and 10 mM HCl to a final concentration of 5% SDS and 5 mM HCl. Finally, the absorbance of the solution was read at 570 nm with a reference at 670 nm in a microplate reader (SpectraMax 340; Molecular Devices, Sunnyvale, Calif.) connected to an Apple Macintosh computer running the program Softmax Pro V1.2.0 (Molecular Devices). Viability is expressed as percent absorbance (A_{570 nm}-A_{670 nm}) relative to the untreated control cells. All values were given as arithmetic means±SD for in vitro cell MTT viability data. In vitro responses of Dx, MnDPDP, and/or DPDP with regard to viability are presented as concentration-effect curves. The biphasic concentration-effect curve of Dx was analyzed by fitting the experimental data into a biphasic sigmoidal four-parameter logistic equation (GraphPad Prism version 5.02). From this analysis, the low and Q7 high pD2 values (negative log of the concentration of Dx that produces half of its maximal inhibition in the two phases, −logIC50) were calculated.

### Results

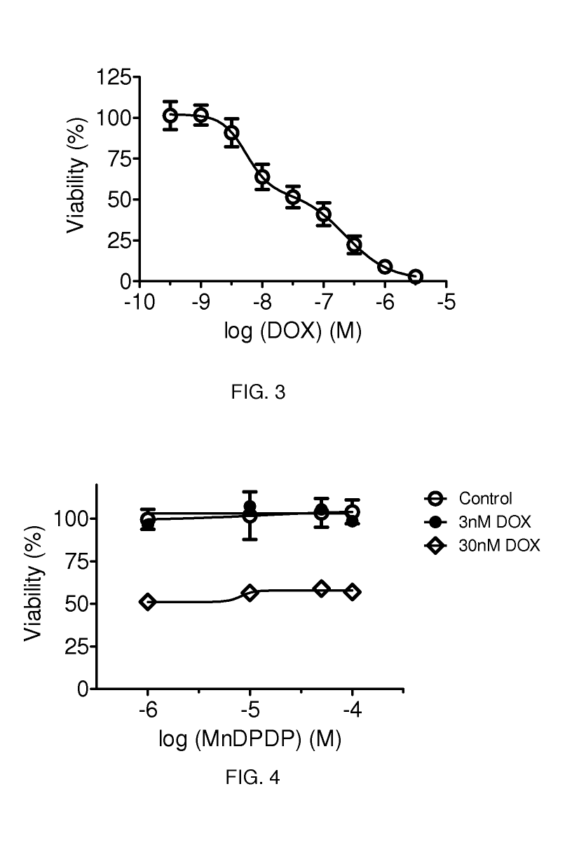

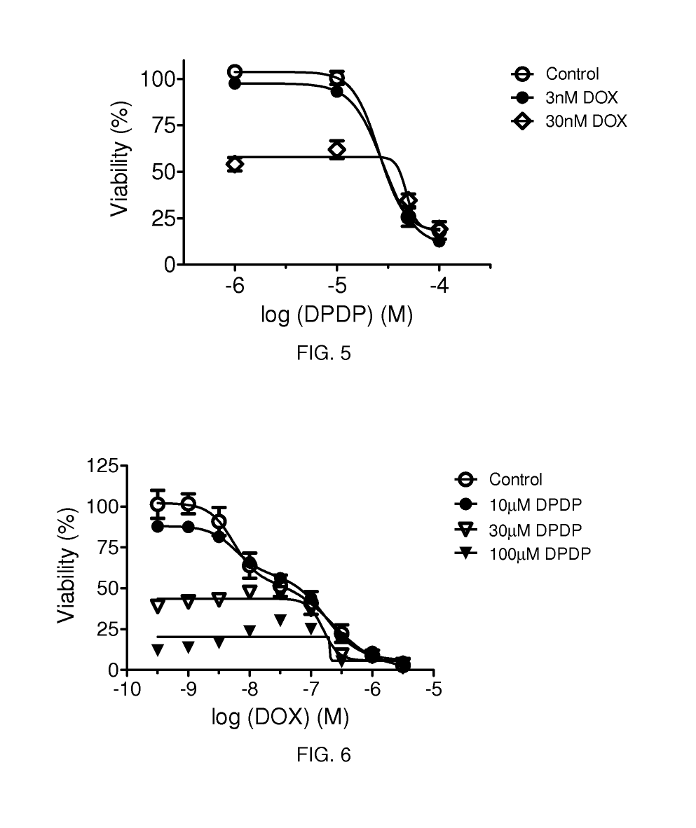

The cytotoxic activity of Dx alone on A2780 cancer cells is presented in FIG. 2. It is apparent that the concentration response curve for A2780 displays more than one phase. When data from subsequent experiments in A2780 cells including some lower Dx concentrations were fitted into a biphasic sigmoidal four parameter logistic equation, FIG. 3, it resulted in two distinct pD₂ (−logEC₅₀) values: 8.264 (95% confidence interval=8.001-8.528) and 6.647 (95% confidence interval=6.273-7.020), respectively. Although results from MTT tests are not necessarily obtained at steady-state conditions, interestingly, the pD₂ values correspond well to the previously described different inhibitory effects of Dx on the topoisomerase II enzyme. MnDPDP, alone or in combination with Dx at a threshold concentration (3 nM) and at a concentration around half-maximal effect (30 nM), did not have any obvious cytotoxic effects in A2780 cells, FIG. 4. Conversely, DPDP alone had cytotoxic effects on A2780 cells, FIG. 5. Surprisingly, neither Dx at threshold concentration nor at a concentration around half-maximal effect revealed any additive effect to the cytotoxic effect of DPDP alone in A2780 cells. One would expect to see a clear additive effect around the half-maximal concentrations of these two compounds. Furthermore, addition of DPDP close to the threshold concentration (10 μM) or the half-maximal concentration (30 μM) did not reveal any obvious additive effect to the cytotoxic effect of Dx alone in A2780, FIG. 6.

### Conclusion

The present results further show efficacy of the presently claimed treatment methods in that DPDP alone displayed cytotoxic activity in A2780 cells, with 30 μM DPDP killing approximately 50% of the cancer cells and 100 μM killing almost all cells.

The specific examples and embodiments described herein are exemplary only in nature and are not intended to be limiting of the invention defined by the claims. Further embodiments and examples, and advantages thereof, will be apparent to one of ordinary skill in the art in view of this specification and are within the scope of the claimed invention.

1. A method for treatment of cancer selected from lung cancer, ovarian cancer, squamous cell carcinoma, pancreas exocrine cancer, malignant melanoma, gastric cancer, esophageal cancer, a metastases thereof, and leukemia, in a human or non-human body, said method comprising administrating to said body a cancer-inhibiting amount of a first compound of Formula I: 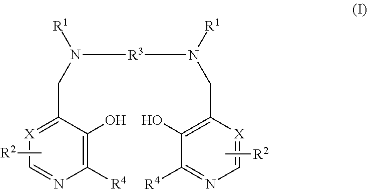
or a physiologically acceptable salt thereof, wherein
X is CH or N,
each R¹ independently is hydrogen or —CH₂COR⁵;
R⁵ is hydroxy, ethylene glycol, glycerol, optionally hydroxylated alkoxy, amino or alkylamido;
each R² independently is a group ZYR⁶;
Z is a bond, CO, or a C₁₋₃ alkylene or oxoalkylene group optionally substituted by a group R⁷;
Y is a bond, an oxygen atom or a group NR⁶;
R⁶ is a hydrogen atom, COOR⁸, an alkyl, alkenyl, cycloalkyl, aryl or aralkyl group optionally substituted by one or more groups selected from COOR⁸, CONR⁸₂, NR⁸₂, OR⁸, ═NR⁸, ═O, OP(O)(OR⁸)R⁷ and OSO₃M;
R⁷ is hydroxy, an optionally hydroxylated, optionally alkoxylated alkyl or aminoalkyl group;
R⁸ is a hydrogen atom or an optionally hydroxylated, optionally alkoxylated alkyl group;
M is a hydrogen atom or one equivalent of a physiologically tolerable cation;
R³ is a C₁₋₈ alkylene group, a 1,2-cykloalkylene group, or a 1,2-arylene group, optionally substituted with R⁷; and
each R⁴ independently is hydrogen or C₁₋₃ alkyl.

2. The method of claim 1, wherein:
R⁵ is hydroxy, C₁₋₈ alkoxy, ethylene glycol, glycerol, amino or C₁₋₈ alkylamido;
Z is a bond or a group selected from CH₂, (CH₂)₂, CO, CH₂CO, CH₂CH₂CO and CH₂COCH₂;
Y is a bond;
R⁶ is a mono- or poly(hydroxy or alkoxylated) alkyl group or a group of the formula OP(O) (OR⁸)R⁷; and
R⁷ is hydroxy, or an unsubstituted alkyl or aminoalkyl group.

3. The method of claim 1, wherein R³ is ethylene and each group R¹ represents
—CH₂COR⁸ in which R⁵ is hydroxy.

4. The method of claim 1, wherein the first compound is N,N′-dipyridoxyl ethylenediamine-N,N′-diacetic acid.

5. The method of claim 1, wherein the first compound is N,N′-bis-(pyridoxal-5-phosphate)-ethylenediamine-N,N′-diacetic acid.

6. The method of claim 1, wherein the cancer is lung cancer and/or metastases thereof.

7. The method of claim 1, wherein the cancer is non-small cell lung cancer and/or metastases thereof.

8. The method of claim 1, wherein the cancer is ovarian cancer and/or metastases thereof.

9. The method of claim 1, wherein the cancer is pancreas exocrine cancer and/or metastases thereof.

10. The method of claim 1, wherein the cancer is malignant melanoma cancer and/or metastases thereof.

11. The method of claim 1, wherein the cancer is gastric cancer and/or metastases thereof.

12. The method of claim 1, wherein the cancer is esophagael cancer and/or metastases thereof.

13. The method of claim 1, wherein the cancer is leukemia.

14. The method of claim 1, wherein the first compound is administered with a cyto-protective amount of a metal chelate of a compound of Formula I.

15. The method of claim 14, wherein said metal chelate has a Kₐ value in the range of from 10⁸ to 10²⁴.

16. The method of claim 14, wherein said metal chelate has a lower Kₐ value than the Kₐ value of an iron (Fe³⁺) chelate of a compound of Formula I, by a factor of at least 10³.

17. The method of claim 14, wherein the metal chelate is a manganese (Mn²⁺ or Mn³⁺) or copper (Cu⁺ or Cu²⁺) chelate.

18. The method of claim 14, wherein the first compound is N, N′-dipyridoxyl ethylenediamine-N,N′-diacetic acid and metal chelate is a metal chelate of N, N′-dipyridoxyl ethylenediamine-N,N′-diacetic acid.

19. The method of claim 14, wherein the first compound is N,N-bis-(pyridoxal-5-phosphate)-ethylenediamine-N,N′-diacetic acid and the metal chelate is a metal chelate of N,N′-dipyridoxyl ethylenediamine-N,N′-diacetic acid.

20. The method of claim 1, wherein the first compound is administered together with one or more other anti-cancer drugs selected from the group consisting of doxorubicin, epirubicin, oxaliplatin, cisplatin, carboplatin, paclitaxel, docetaxel, 5-fluorouracil, cyclophosphamide, gemcitabine, irinotecan, and methotrexate.

21. The method of claim 20, wherein the first compound and the one or more other anti-cancer drug(s) are administered simultaneously, separately or sequentially to said patient.

22. The method of claim 1, wherein the first compound is administered in combination with radiation therapy.

23. The method of claim 5, wherein the cancer is lung cancer and/or metastases thereof.

24. The method of claim 5, wherein the cancer is non-small cell lung cancer and/or metastases thereof.

25. The method of claim 5, wherein the cancer is ovarian cancer and/or metastases thereof.

26. The method of claim 5, wherein the cancer is pancreas exocrine cancer and/or metastases thereof.

27. The method of claim 5, wherein the cancer is malignant melanoma cancer and/or metastases thereof.

28. The method of claim 5, wherein the cancer is gastric cancer and/or metastases thereof.

29. The method of claim 5, wherein the cancer is esophagael cancer and/or metastases thereof.

30. The method of claim 5, wherein the cancer is leukemia.
